(19) United States
(12) Patent Application Publication
(10) Pub. No.: US 2009/0042041 A1
(43) Pub. Date: Feb. 12, 2009

(54) PYRROLYL COMPLEXES OF COPPER FOR COPPER METAL DEPOSITION

(75) Inventor: Vladimir Grushin, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. duPont de Nemours and Company, Wilmington, DE (US)

(21) Appl. No.: 12/254,465

(22) Filed: Oct. 20, 2008

Related U.S. Application Data
(63) Continuation-in-part of application No. 10/523,493, filed on Feb. 3, 2005.

(51) Int. Cl. B32B 17/06; C07D 207/335; C07F 1/08; B32B 9/04; B32B 15/04; B05D 3/10

(52) U.S. Cl. 428/432; 548/561; 548/402; 427/343; 428/457; 428/446

(57) ABSTRACT

The present invention relates to a process for the preparation of ligands and copper complexes useful in the deposition of copper via Atomic Layer Deposition (ALD) and Chemical Vapor Deposition (CVD), and the use of the copper complexes in ALD and CVD processes.

### FIELD OF THE INVENTION

The present invention relates to a process for the preparation of ligands and copper complexes useful in the deposition of copper via Atomic Layer Deposition or Chemical Vapor Deposition.

### TECHNICAL BACKGROUND

Atomic Layer Deposition (ALD) and Chemical Vapor Deposition (CVD) processes are useful in the deposition of metals on substrates.

In the ALD process, a substrate on which the metal is to be deposited is placed in a vacuum chamber. A volatile metal complex is then admitted into the vacuum chamber and allowed to adsorb onto a substrate. The excess, unadsorbed vapor of the metal complex is then pumped or purged from the vacuum chamber. The adsorbed metal complex is then exposed to a second reagent, which causes the complex to react to produce metal. In the preparation of a copper film from a copper(II) complex, the second reagent is a reducing agent. Suitable copper precursor complexes for this process must be volatile enough to sublime and thermally stable in the temperature range of the process. The ligands themselves should preferably leave as the free ligand.

In a CVD process, a heated substrate is exposed to a vapor of the volatile metal complex, optionally in the presence of another reactant (a co-reactant) in the gas phase. In this process, the complex decomposes to metal on contact with the substrate, or reacts with the co-reactant(s) in the vicinity of the substrate, to produce a deposited metal film. The ligand can evolve either as volatile free ligand or be decomposed into volatile by-products. Thus, for CVD, the metal complex must be volatile and stable enough to form a vapor phase, but unlike for ALD, must decompose on contact with the heated substrate in the absence or presence of a co-reactant under the conditions of the CVD process to give the desired film.

Copper films formed via ALD or CVD processes are useful in many applications, including the production of electronic devices, catalytic surfaces and decorative effects.

K. Yeh and R. H. Parker, Inorganic Chemistry, 6, 830-833 (1967), disclose the synthesis of 2-pyrrolealdimines and the corresponding copper chelates. B. Emmert, et al., Berichte, 62, 1733-1738 (1929), disclose the synthesis of 2-pyrrolealdmethylimine and of its copper complex.

### SUMMARY OF THE INVENTION

The present invention relates to processes for the preparation of ligands and the corresponding copper complexes, and the use of such complexes to deposit copper onto substrates.

A first embodiment of this invention relates to a process for preparing pyrrolealdimines, comprising the steps of:
a) reacting 2-formylpyrrole with a primary amine, RNH₂, in an aqueous solution at a temperature of 5-30° C., wherein R is C₁ to C₁₀ alkyl or substituted alkyl, or C₆ to C₁₂ aryl or substituted aryl;
b) adding a water-immiscible organic compound to form an aqueous phase and an organic phase; and
c) isolating the organic phase.

A second embodiment of this invention is an aqueous process for preparing Cu(II) complexes of 2-pyrrolyl imino ligands comprising reacting an aqueous mixture of 2-formylpyrrole and a source of Cu(II) with a primary amine, R¹NH₂, wherein

R¹ is selected from the group consisting of C₁-C₁₀ alkyl or substituted alkyl; C₆ to C₁₂ aryl or substituted aryl; allyl; benzyl; NHR³; and NR⁴R⁵; and

R³, R⁴, and R⁵ are independently selected from C₁-C₆ alkyl or substituted alkyl and C₆ to C₁₂ aryl or substituted aryl.

A third embodiment of this invention is a process for preparing Cu(II) complexes of a 2-acylpyrrole comprising:
a) combining a source of Cu(II), water, and a 2-acylpyrrole,

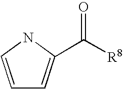

where R⁸ is C₁ to C₁₀ alkyl, to form an aqueous mixture; and
b) adding a base to the aqueous mixture.

A fourth embodiment of this invention provides Cu(II) complexes comprising:
a) a copper atom; and
b) two pyrrole ligands bound to said copper atom, wherein said pyrrole ligands are independently selected from the group consisting of 2-pyrroleald-n-propylimino, 2-pyrroleald-1-butyl-imino, 2-pyrroleald-n-butyl-imino, 2-pyrroleald-2-ethylhexyl-imino, 2-pyrroleald-m-trifluoromethylphenyl-imino, 2-pyrrolylald(2-isopropoxycarbonylethyl)imino, 2-pyrrolylald(2-ethoxycarbonylethyl)imino and 2-pyrroleald-benzyl-imino ligands.

A fifth embodiment of this invention is a process for depositing copper on a substrate comprising:
a) adsorbing onto a substrate at least one Cu(II) complex of structure 1

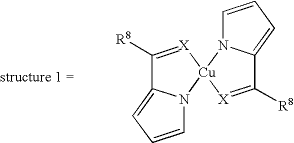

wherein:

X is O, and R⁸ is C₁-C₁₀ alkyl or substituted alkyl, or C₆ to C₁₂ aryl or substituted aryl; or

X is NR¹ and R⁸ is H;

R¹ is selected from the group comprising C₁-C₁₀ alkyl or substituted alkyl; C₆ to C₁₂ aryl or substituted aryl; allyl; benzyl; NHR³; and NR⁴R⁵; and

R³, R⁴, and R⁵ are independently selected from C₁-C₆ alkyl or substituted alkyl, and C₆ to C₁₂ aryl or substituted aryl; and
b) exposing the absorbed complex to a reducing agent to form copper metal.

A sixth embodiment of this invention provides a process for depositing copper on a substrate, comprising heating in the presence of a substrate a reducing agent and at least one Cu(II) complex of structure 1,

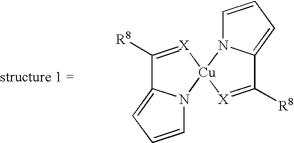

wherein:

X is O, and R⁸ is C₁-C₁₀ alkyl or substituted alkyl, or C₆ to C₁₂ aryl or substituted aryl; or

X is NR¹ and R⁸ is H;

R¹ is selected from the group consisting of C₁-C₁₀ alkyl or substituted alkyl; C₆ to C₁₂ aryl or substituted aryl; allyl; benzyl; NHR³; and NR⁴R⁵; and

R³, R⁴, and R⁵ are independently chosen from C₁-C₆ alkyl or substituted alkyl, and C₆ to C₁₂ aryl or substituted aryl.

A seventh embodiment of this invention is an article comprising a substrate with a Cu(II) complex of structure 1 adsorbed on the surface or in or on porosity in the substrate,

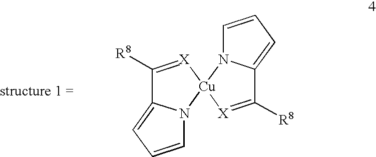

wherein:

X is O, and R⁸ is C₁-C₁₀ alkyl or substituted alkyl, or C₆ to C₁₂ aryl or substituted aryl; or

X is NR¹ and R⁸ is H;

R¹ is selected from the group consisting of C₁-C₁₀ alkyl or substituted alkyl; C₆ to C₁₂ aryl or substituted aryl; allyl; benzyl; NHR³, and NR⁴R⁵; and

R³, R⁴, and R⁵ are independently selected from C₁-C₆ alkyl or substituted alkyl, and C₆ to C₁₂ aryl or substituted aryl.

### DETAILED DESCRIPTION

It has been found that pyrrolealdimino and pyrroleketo complexes of copper are especially useful as volatile copper precursors for ALD and/or CVD processes. These complexes are air- and moisture-stable, thermally stable and volatile under ALD and/or CVD process conditions. These complexes can be decomposed in the presence of appropriate reducing agents to form copper metal.

The pyrrolealdimino and pyrroleketo copper (II) complexes are easily prepared in good yield in aqueous media from readily available reagents. For example, the pyrrolealdimine ligands can be isolated from the reaction of 2-formylpyrrole with the appropriate primary amine, and then reacted with a source of copper (II) to give the desired copper (II) complex. Alternatively, the pyrrolealdimine ligand can be made in situ, such that the pyrrolealdimino copper complex is isolated directly from the aqueous reaction mixture of 2-formylpyrrole, the primary amine and a source of copper(II).

A preferred method for preparing the pyrrolealdimine ligands is to react 2-formylpyrrole with a primary amine in water at room temperature, either by adding an aqueous solution of the amine to the 2-formylpyrrole, or by adding the amine to a mixture of water and 2-formylpyrrole. Then a water-immiscible organic compound is added to form a two-phase system in which the pyrrolealdimine ligand is extracted into the organic phase. Alternatively, an aqueous solution of the amine is added to a suspension of 2-formylpyrrole in a water-immiscible solvent. Upon reaction, the pyrrolealdimine ligand is extracted into the water-immiscible phase. Preferred primary amines, RNH₂, are those for which R is C₁ to C₁₀ alkyl or substituted alkyl, or C₆ to C₁₂ aryl or substituted aryl. In some embodiments, R is methyl or phenyl. In some embodiments R is C₁ to C₆ alkyl or substituted alkyl. The preferred molar ratio of 2-formylpyrrole to primary amine is from about 1:2 to 2:1. In one embodiment, the temperature is about 5° C. to about 30° C., or the temperature is about 10° C. and about 25° C. Preferred water-immiscible compounds are organic solvents such as alkanes, chlorinated alkanes, cycloalkanes, and aromatic solvents. Especially preferred solvents include pentane, hexanes, heptanes, chloroform, dichloromethane, carbon tetrachloride, cylcopentane, cyclohexane, benzene, and toluene. The pyrrolealdimine ligand can be isolated from the organic solvent by conventional means and further purified, if necessary, by crystallization, sublimation or other common methods. Alternatively, the water-immiscible organic compound is a water-immiscible liquid primary amine which functions as both the reagent and the organic phase.

The desired copper(II) complexes can also be obtained by reacting 2-formylpyrrole, a primary amine, R¹NH₂, and a source of copper(II) in water. Suitable primary amines are of the form, R¹NH₂, where R¹ is selected from the group comprising C₁-C₁₀ alkyl or substituted alkyl; C₆ to C₁₂ aryl or substituted aryl; allyl; benzyl; NHR³; and NR⁴R⁵, wherein R³, R⁴, and R⁵ are independently selected from C₁-C₆ alkyl or substituted alkyl, and C₆ to C₁₂ aryl or substituted aryl. In some embodiments, R¹ is methyl or phenyl. In some embodiments R¹ is C1 to C6 alkyl or substituted alkyl.

Suitable substituent groups on the substituted alkyls and substituted aryls include F, Cl, perfluoroalkyls, alkyl esters, methoxy and ethoxy groups. For some electronic applications, complexes containing only Cu, C, H, and N are preferred.

The preferred molar ratio of 2-formylpyrrole to primary amine is from about 1:1 to about 1:10. The preferred molar ratio of copper to 2-formylpyrrole is from about 10 to 1 to about 1 to 10, more preferably from about 1.2 to 2. Preferred temperatures are about 0° C. to about 100° C., more preferably about 20° C. to about 80° C. If the product is a solid, it may be isolated and purified by standard methods (e.g., filtration, recrystallization, sublimation, etc.). If the product is an oil, it may be isolated by decanting off the aqueous phase, and then purified by standard methods (e.g., chromatography or distillation).

Alternatively, 2-formylpyrrole and a primary amine are reacted in water, and then a source of copper(II) is added and the resulting mixture is allowed to react to form the copper(II) complex. Isolation of the complex is carried out as described above.

Alternatively, the synthesis of the copper pyrrolylaldimino complexes may be performed under biphasic conditions, i.e., in the presence of an organic solvent that is immiscible with water, yet capable of dissolving the desired Cu complex product. In this process, the Cu complex product will be extracted, fully or partially, into the organic phase as it forms. The biphasic technique may be beneficial due to more efficient agitation of the reaction mixture and higher conversions after shorter reaction times. After the reaction, the desired Cu complex product can be isolated from the organic phase via conventional filtration, evaporation, and recrystallization (if necessary). Solvents suitable for this technique include dichloromethane, toluene, benzene, ether, alkanes, and cycloalkanes, as long as the particular complex product exhibits sufficient solubility in such media.

The products of the reactions of 2-formylpyrrole, a primary amine, R¹NH₂ and a source of copper(II) are bis(2-pyrrolealdimino)copper(II) complexes in which the imino nitrogen is substituted with R¹ selected from the group consisting of C₁-C₁₀ alkyl or substituted alkyl; C₆ to C₁₂ aryl or substituted aryl; allyl; benzyl; NHR³; and NR⁴R⁵; where R³, R⁴, and R⁵ are independently selected from C₁-C₆ alkyl or substituted alkyl, and C₆ to C₁₂ aryl or substituted aryl.

Bis(acylpyrrolyl)copper(II) complexes can be prepared by reacting an acylpyrrole and a source of copper(II) in water, followed by addition of a base. Suitable 2-acylpyrroles include acetylpyrrole. Suitable bases include NaOH, KOH, and calcium hydroxide. Freshly precipitated copper(II) hydroxide may be used in the absence of extra base. The 2-acylpyrroles useful in this process are readily synthesized using known procedures.

Suitable sources of copper(II) include copper hydroxide, copper(II) chloride, copper nitrate, copper sulfate, copper(II) salts of carboxylic acids (e.g., copper acetate and copper benzoate), and copper alkoxides (e.g., copper methoxide). Either the hydrated or the anhydrous form of these copper(II) salts may be used. If a copper(II) salt of a strong acid is used (such as nitrate, sulfate, chloride, and bromide) the acid which is released during the synthesis may be neutralized by addition of a base such as alkali (NaOH, KOH, Ba(OH)₂) or Ca(OH)₂, or any base that can scavenge the acid while not interfering with Cu complex formation

This invention includes compositions comprising a copper atom coordinated to two bidentate pyrrole ligands. The pyrrole ligand has either an acyl or aldimine group in the 2-position of the pyrrole ring. The ligands are chosen to form a copper(II) complex that is volatile in an appropriate temperature range (typically 20° C. to 250° C.) but does not decompose in this temperature range; however, the complex decomposes to metal on addition of a suitable reducing agent. The ligand is further chosen so that the ligand and or products of its transformations will desorb upon exposure to a reducing agent during the atomic layer deposition process.

The copper(II) complexes of this invention are suitable for use in ALD and CVD processes for creation of copper films for use as seed layers in formation of copper interconnects on integrated circuits or as decorative or catalytic applications.

In an ALD process of this invention, a substrate on which copper is to be deposited is placed in a vacuum chamber. At least one copper(II) complex (I) is then admitted into the vacuum chamber and allowed to adsorb onto the substrate. The copper complex will be added to a reactor at a temperature, time and pressure to attain a suitable fluence of complex to the surface. One of skill in the art will appreciate that the selection of these variables will depend on individual chamber and system design and the desired process rate. The excess, unadsorbed vapor of the copper complex is then pumped or purged from the vacuum chamber. The adsorbed metal complex is then exposed to a reducing reagent at a pressure of approximately 10 to 760 millitorr, which causes the complex to decompose to copper and free ligand. The substrate is held at a temperature of about 50° C. to 300° C. during reduction. Reducing agent exposure times may be from about a second to several hours. Finally, the ligand and/or the products of its transformation are removed by evacuation of the chamber.

In a CVD process, the copper film is produced when the vapor of a volatile copper(II) complex decomposes on contact with a heated substrate. A gas-phase reducing agent can be added with the volatile copper complex to facilitate the clean decomposition of the complex. In a CVD process, the substrate is heated to approximately 100° C. to 300° C. The ligand and/or the products of its decomposition are removed by evacuation of the chamber or an inert gas sweep.

Suitable substrates for the ALD and CVD processes include glass, metals and ceramics, preferably silicon wafers coated with a barrier layer such as titanium nitride or tantalum/tantalum nitride.

Suitable reducing reagents for the ALD and CVD processes of this invention include ammonia and ammonia/hydrogen mixtures, hydrazine, CO/hydrogen mixtures, 9-BBN, borane, dihydrobenzofuran, pyrazoline, diethylsilane, dimethylsilane, ethylsilane, phenylsilane, and silane. Ammonia/hydrogen mixtures and diethylsilane are preferred.

Preferred copper (II) complexes for use in the ALD and CVD processes of this invention include bis(2-pyrrolylaldmethylimino)copper(II), bis(2-pyrrolylaldethylimino)copper(II), bis(2-pyrrolylald-n-propylimino)copper(II), bis(2-pyrrolylaldisopropylimino)copper(II), bis(2-pyrrolylald-n-butylimino)copper(II), bis(2-pyrrolylald-t-butylimino)copper(II), bis(2-pyrrolylaldisobutylimino)copper(II), bis(2-pyrrolylaldphenyl imino)copper(II), bis(2-pyrrolylald(m-trifluoromethylphenyl)imino)copper(II), bis(2-pyrrolylaldbenzylimino)copper(II), and bis(2-acetylpyrrolyl)copper(II).

### EXAMPLES

Unless otherwise specified, all temperatures are in degrees Celsius, all mass measurements are in grams, and all solution percentages are weight percentages.

### Preparation of Ligands

### Example 1

2-Pyrrolealdmethylimine. Aqueous methylamine (40%; 4 mL) was added, with stirring, to 2-formylpyrrole (2.00 g). After stirring for 1 min at room temperature, a white solid formed. Water (25 mL) and hexanes (150 mL) were added with stirring to produce a solids-free liquid-liquid biphasic mixture. After 15 min of stirring the organic layer was separated and filtered through a short column filled with anhydrous sodium sulfate. The clear, colorless filtrate was reduced in volume to about 20 mL and kept at +5° C. overnight. The white crystals were collected, dried with a nitrogen flow (the compound is very volatile), and sublimed under vacuum to give 1.80 g (79%) of white 2-pyrrolealdmethylimine (identical with an authentic sample).

### Example 2

2-Pyrrolealdphenylimine. A mixture of 2-formylpyrrole (5.00 g), aniline (5 mL), and water (50 mL) was vigorously stirred at room temperature in air for 3 days. The resulting solid was filtered, washed with water, and dried under vacuum to give 8.45 g (94%) of spectroscopically pure (¹H NMR) 2-pyrrolealdphenylimine (identical with an authentic sample).

### Example 3

2-Pyrroleald(t-butyl)imine. t-Butylamine (2.35 g) was added to 2-formylpyrrole (2.00 g). After the latter had all dissolved upon gentle swirling, water (20 mL) and hexanes (50 mL) were added. The mixture was stirred until two clear, non-cloudy liquid layers were formed (ca. 30 min). The organic layer was separated, filtered through a short anhydrous Na₂SO₄ plug, and evaporated to leave a viscous colorless oil. The oil crystallized after 2 h at +5° C. and the solid stayed crystalline after warming up to room temperature. The yield was 2.98 g (94%). The compound was identified by its ¹H NMR spectrum indistinguishable from that reported in Inorg. Chim. Acta, 1984, vol. 89, p. 79.

### Example 4

2-Pyrrolealdmethylimine. Aqueous methylamine (40%; 10 mL) was added to a stirred suspension of 2-formylpyrrole (5.22 g) in hexanes (150 mL). After 15 min of stirring, the upper organic layer was separated and filtered through a short column filled with anhydrous sodium sulfate. The column was washed with hexanes. The combined hexane filtrates were evaporated to leave 5.75 g (97%) of the crude product as slightly yellowish crystals. The latter were sublimed under vacuum to produce 5.35 g (90%) of white 2-pyrrolealdmethylimine (identical with an authentic sample). The compound turns yellow and eventually dark brown, even if stored under nitrogen.

### Example 5

2-Pyrrolealdethylimine. Aqueous ethylamine (70%; 3.6 mL) was added, with stirring, to 2-formylpyrrole (2.1 g). The solid all dissolved within 1 min of stirring. After stirring for 5 min, the viscous solution turned cloudy due to phase segregation. Water (25 mL) and hexanes (60 mL) were added with stirring to produce a colorless, solid-free, liquid-liquid biphasic mixture. After 15 min of stirring, the organic layer was separated and filtered through a short column filled with anhydrous Na₂SO₄. The aqueous phase was washed with hexanes (2×10 mL), and the extracts were used to wash the Na₂SO₄ column. The combined colorless filtrates were evaporated under nitrogen to leave, after quickly drying under vacuum, 2.56 g (95%) of 2-pyrrolealdethylimine as spectroscopically pure, white crystals. The compound turns yellow even if stored under nitrogen.

¹H NMR (CD₂Cl₂, 20° C.), δ: 1.3 (t, 3H, J=7.3 Hz, CH₃); 3.65 (dq, 2H, J=7.3 and 1.2 Hz, CH₂); 6.3 (dd, 1H, J=2.7 and 3.5 Hz, pyrr-CH); 6.6 (dd, 1H, J=3.5 and 1.2 Hz, pyrr-CH); 7.0 (m, 1H, pyrr-CH); 8.1 (br m, 1H, imine CH); 11.0 (br s, 1H, NH). ¹³C NMR (CD₂Cl₂, 20° C.), δ: 16.7; 55.2; 109.8; 114.7; 122.4; 130.9; 152.2.

### Example 6

2-Pyrrolealdpropylimine. Propylamine (4.2 mL) was added, with stirring, to a mixture of 2-formylpyrrole (2.42 g) and water (5 mL). The solid quickly dissolved. After stirring for 5 min, a liquid-liquid biphasic system formed. Water (15 mL) and hexanes (25 mL) were added, and the mixture was vigorously stirred for 15 min. The organic layer was separated and filtered through a short column filled with anhydrous Na₂SO₄. The aqueous phase was washed with hexanes (3×10 mL), and the extracts were used to wash the Na₂SO₄ column. The combined colorless filtrates were evaporated under nitrogen to leave, after quickly drying under vacuum, 3.35 g (97%) of 2-pyrrolealdpropylimine as a spectroscopically pure, slightly yellowish oil. ¹H NMR (CD₂Cl₂, 20° C.), δ: 0.95 (t, 3H, J=7.3 Hz, CH₃); 1.7 (m, 2H, CH₂); 3.5 (m, 2H, CH₂); 6.25 (dd, 1H, J=2.7 and 3.3 Hz, pyrr-CH); 6.5 (dd, 1H, J=3.3 and 1.2 Hz, pyrr-CH); 6.9 (m, 1H, pyrr-CH); 8.1 (br m, 1H, imine CH). ¹³C NMR (CD₂Cl₂, 20° C.), δ: 11.9; 24.7; 63.0; 109.8; 113.8; 121.7; 130.9; 151.7.

### Example 7

2-Pyrrolealdisoprolylimine. Isopropylamine (4.7 mL) was added, with stirring, to a mixture of 2-formylpyrrole (2.6 g) and water (4 mL). The solid dissolved within 1 min, and then a milky emulsion formed. After stirring for 5 min, water (20 mL) and hexanes (25 mL) were added, and the mixture was vigorously stirred for 15 min. The organic layer was separated and filtered through a short column filled with anhydrous Na₂SO₄. The aqueous phase was washed with hexanes (3×10 mL), and the extracts were used to wash the Na₂SO₄ column. The combined colorless filtrates were evaporated under nitrogen to leave, after quickly drying under vacuum, 3.6 g (97%) of 2-pyrrolealdisopropylimine as a spectroscopically pure, slightly yellowish oil. ¹H NMR (CD₂Cl₂, 20° C.), δ: 1.2 (d, 6H, J=6.4 Hz, CH₃); 3.45 (sept, 1H. J=6.4 Hz, CHCH₃); 6.2 (dd, 1H, J=2.8 and 3.5 Hz, pyrr-CH); 6.45 (dd, 1H, J=3.5 and 1.5 Hz, pyrr-CH); 6.9 (m, 1H, pyrr-CH); 8.1 (s, 1H, imine CH). ¹³C NMR (CD₂Cl₂, 20° C.), δ: 24.6; 61.3; 109.8; 114.2; 122.1; 131.0; 150.2.

### Example 8

2-Pyrrolealdbutylimine. Butylamine (5.2 mL) was added, with stirring, to a mixture of 2-formylpyrrole (2.5 g) and water (5 mL). After stirring for 5 min, water (15 mL) and hexanes (25 mL) were added, and the mixture was vigorously stirred for 15 min. The organic layer was separated and filtered through a short column filled with anhydrous Na₂SO₄. The aqueous phase was washed with hexanes (3×10 mL), and the extracts were used to wash the Na₂SO₄ column. The combined colorless filtrates were evaporated under nitrogen to leave a colorless oil which crystallized on drying under vacuum. The yield of 2-pyrrolealdbutylimine as a spectroscopically pure, white crystalline solid was 3.9 g (99%). ¹H NMR (CD₂Cl₂, 20° C.), δ: 1.0 (t, 3H, J=7.3 Hz, CH₃); 1.4 (m, 2H, CH₂); 1.65 (m, 2H, CH₂); 3.6 (dt, 2H, J=7.0 and 1.2 Hz, CH₂); 6.2 (dd, 1H, J=2.8 and 3.7 Hz, pyrr-CH); 6.5 (dd, 1H, J=3.7 and 1.5 Hz, pyrr-CH); 6.9 (m, 1H, pyrr-CH); 8.1 (m, 1H, imine CH); 10.0 (br s, 1H, NH). ¹³C NMR (CD₂Cl₂, 20° C.), δ: 14.0; 20.7; 33.7; 60.8; 109.7; 114.3; 122.1; 130.8; 152.3.

### Example 9

2-Pyrrolealdisobutylimine. Isobutylamine (4.8 mL) was added, with stirring, to a mixture of 2-formylpyrrole (2.3 g) and water (4 mL). After stirring for 2 min, water (15 mL) and hexanes (25 mL) were added, and the mixture was vigorously stirred for 15 min. The organic layer was separated and filtered through a short column filled with anhydrous Na₂SO₄. The aqueous phase was washed with hexanes (3×15 mL), and the extracts were used to wash the Na₂SO₄ column. The combined colorless filtrates were evaporated under nitrogen to leave, after drying under vacuum, 3.6 g (99%) of 2-pyrrolealdisobutylimine as a spectroscopically pure, white crystalline solid. The product turns yellow even if stored under nitrogen. Anal. Calcd. for C₉H₁₄N₂, %: C, 72.0; H, 9.4; N, 18.6. Found, %: C, 72.4; H, 9.1; N, 18.8. ¹H NMR (CD₂Cl₂, 20° C.), δ: 0.95 (d, 6H, J=6.7 Hz, CH₃); 1.9 (sept, 1H, J=6.7 Hz, Me₂CH); 3.45 (m, 2H, NCH₂); 6.2 (m, 1H, pyrr-H); 6.5 (m, 1H, pyrr-H); 6.9 (m, 1H, pyrr-H); 8.1 (s, 1H, imine CH); 10.0 (br s, 1H, NH). ¹³C NMR (CD₂Cl₂, 20° C.), δ: 20.7; 30.2; 69.3; 109.7; 114.2; 122.0; 130.8; 152.4.

### Example 10

2-Pyrroleald-sec-butylimine. sec-Butylamine (5.1 mL) was added, with stirring, to a mixture of 2-formylpyrrole (2.4 g) and water (5 mL). After stirring for 5 min, water (15 mL) and hexanes (25 mL) were added, and the mixture was vigorously stirred for 15 min. The organic layer was separated and filtered through a short column filled with anhydrous Na₂SO₄. The aqueous phase was washed with hexanes (3×15 mL), and the extracts were used to wash the Na₂SO₄ column. The combined colorless filtrates were evaporated under nitrogen and the residue dried under vacuum to leave 3.65 g (96%) of 2-pyrroleald-sec-butylimine as a spectroscopically pure, slightly yellowish oil. Anal. Calcd. for C₉H₁₄N₂, %: C, 72.0; H, 9.4; N, 18.6. Found, %: C, 71.1; H, 9.1; N, 18.7. ¹H NMR (CD₂Cl₂, 20° C.), δ: 0.9 (t, 3H, J=7.6 Hz, CH₃); 1.3 (d, 3H, J=6.4 Hz; CH₃); 1.6 (m, 2H, CH₂); 3.2 (m, 1H, CH); 6.25 (dd, 1H, J=2.7 and 3.6 Hz, pyrr-CH); 6.5 (dd, 1H, J=3.6 and 1.2 Hz, pyrr-CH); 6.9 (m, 1H, pyrr-CH); 8.1 (s, 1H, imine CH); 10.6 (br s, 1H, NH). ¹³C NMR (CD₂Cl₂, 20° C.), δ: 11.2; 22.7; 31.1; 67.8; 109.7; 114.0; 121.9; 130.9; 150.6.

### Preparation of Complexes

### Example 11

Bis(2-pyrrolylaldmethylimino)copper(II). A mixture of 2-formylpyrrole (1.07 g), water (25 mL), and 40% aqueous methylamine (2 mL) was stirred for 1-2 min to produce a milky reaction mixture which quickly turned into a biphasic emulsion. Copper (II) acetate (1.20 g) was added, and the mixture was stirred at room temperature in air for 1 day. The resulting brown solid was filtered, washed with water, air-dried on the filter, and dissolved in dichloromethane. The solution was filtered, reduced in volume to 3-5 mL (dark crystals began to form), and treated with hexanes (25 mL). After 1.5 h at +5° C., the dark crystals were collected, washed with hexanes (3×5 mL), and dried under vacuum. The yield was 1.18 g (75%), m.p. 168-169° C.

### Example 12

Bis(2-pyrrolylaldethylimino)copper(II). This complex (m.p. 131-132° C.) was prepared similarly, using commercially available 70% aqueous EtNH₂ (1.75 mL), 2-formylpyrrole (1.50 g), and Cu (II) acetate (1.72 g) in water (50 mL).

### Example 13

Bis(2-pyrrolylald-n-propylimino)copper(II). Propylamine (2.3 mL) was added, with stirring, to a mixture of 2-formylpyrrole (1.23 g) and water (25 mL). After 1-2 min, when all aldehyde dissolved and a liquid-liquid biphasic system formed, Cu(OAc)₂ (1.40 g) was added. After 20 min of agitation, water (25 mL) was added and the mixture was vigorously stirred at room temperature in air for 4 h. The resulting brown solid was filtered, washed with water, air-dried, and dissolved in dichloromethane. The solution was filtered, reduced in volume to 1-2 mL, and treated with hexanes (10 mL). After keeping the mixture for 1.5 h at +5° C., the black crystals were collected, washed quickly with cold hexanes (2×2 mL), and dried under vacuum. The yield was 1.96 g (91%), m.p. 118-120° C. Anal. Calcd. for C₁₆H₂₂CuN₄, %: C, 57.6; H, 6.6; N, 16.8. Found, %: C, 57.6; H, 6.5; N, 16.7. The structure was confirmed by single-crystal X-ray diffraction.

### Example 14

Bis(2-pyrrolylaldisopropylimino)copper(II). Isopropylamine (2.0 mL) was added, with stirring, to a mixture of 2-formylpyrrole (1.06 g) and water (10 mL). After 2 min, Cu(OAc)₂ (1.20 g) was added. The mixture was stirred at room temperature in air for 1.5 h, then water (50 mL) was added and the stirring continued for 2 more hours. The resulting brown solid was filtered, washed with water, air-dried, and dissolved in dichloromethane. The dark-green solution was filtered and evaporated to leave well-shaped black crystals. The product was recrystallized by dissolving in boiling heptane and then cooling the solution to +5° C. The large black crystals were washed quickly with hexanes (3×3 mL), and dried under vacuum. The yield was 1.71 g (92%), m.p. 124-125° C.

### Example 15

Bis(2-pyrrolylald-n-butylimino)copper(III). n-Butylamine (3.0 mL) was added, with stirring, to a mixture of 2-formylpyrrole (1.38 g) and water (25 mL). After 5 min, Cu(OAc)₂ (1.60 g) was added. The mixture was stirred at room temperature in air for 3.5 h, then water (25 mL) was added and the stirring continued for 3 more hours. The brown solid was filtered, washed with water, air-dried, and dissolved in dichloromethane. The solution was filtered and evaporated to dryness. Methanol (10 mL) was added to the residue and the dark solution was kept at about 5° C. for 4 hours. The well-shaped black crystals were separated, quickly washed with cold methanol, and dried under vacuum. The yield was 2.32 g (88%), m.p. 93-95° C. Anal. Calcd. for C₁₈H₂₆CuN₄, %: C, 59.7; H, 7.2; N, 15.5.

Found, %: C, 59.8; H, 7.1; N, 15.5.

### Example 16

Bis(2-pyrrolylald-t-butylimino)copper(II). t-Butylamine (2.0 mL) was added, with stirring, to a mixture of 2-formylpyrrole (1.00 g) and water (25 mL). After 5 min, Cu(OAc)₂ (1.20 g) was added. The mixture was stirred at room temperature in air for 1 h, then water (25 mL) was added and the stirring continued overnight. The resulting green solid was filtered, washed with water, air-dried, and dissolved in dichloromethane. The solution was filtered and evaporated to dryness. Hexanes (10 mL) were added to the residue and the mixture was kept at ca. 5° C. overnight. The black-green crystals were separated, quickly washed with cold hexanes, dried under vacuum. The yield was 0.88 g (46%), m.p. 153-156° C. The structure was confirmed by single-crystal X-ray diffraction.

### Example 17

Bis(2-pyrrolylaldisobutylimino)copper(II). i-Butylamine (2.7 mL) was added, with stirring, to a mixture of 2-formylpyrrole (1.30 g) and water (15 mL). After 2 min, Cu(OAc)₂ (1.50 g) was added. The mixture was stirred at room temperature in air for 2.5 h, then water (35 mL) was added and the stirring continued for 3 more hours. The resulting solid was filtered, washed with water, air-dried, and dissolved in dichloromethane. The solution was filtered, evaporated to about 3-5 mL, and treated with hexanes (20 mL). After 3 hours at about 5° C., the black crystals were separated, washed with hexanes (3×5 mL), and dried under vacuum. The yield was 2.07 g (84%), m.p. 194-197° C. Anal. Calcd. for C₁₈H₂₆CuN₄, %: C, 59.7; H, 7.2; N, 15.5. Found, %: C, 59.9; H, 7.2; N, 15.5.

### Example 18

Bis(2-pyrrolylald(2-ethylhexyl)imino)copper(II). A mixture of 2-ethylhexylamine (3.3 g), 2-formylpyrrole (2.20 g), water (50 mL), and Cu(OAc)₂ (2.50 g) was stirred at room temperature in air for 3 days. The aqueous phase was decanted off of the dark viscous oil which was then thoroughly washed with water, dissolved in dichloromethane, and filtered through a short silica gel plug. The filtrate was evaporated to leave a dark oil which was dried under vacuum overnight. The yield was 3.65 g (66%). Attempts to obtain a crystalline sample of the product from hexanes or methanol were unsuccessful. Anal. Calcd. for C₂₆H₄₂CuN₄, %: C, 65.9; H, 8.9; N, 11.8. Found, %: C, 65.7; H, 8.9; N, 11.8.

### Example 19

Bis(2-pyrrolylaldphenylimino)copper(II). A mixture of 2-formylpyrrole (3.00 g), water (50 mL), and aniline (3.00 mL) was stirred for 1-2 min. Copper (II) acetate (3.00 g) was added, and the mixture was stirred at room temperature in air overnight. The resulting brown solid was filtered, washed with water, air-dried on the filter, and dissolved in dichloromethane. The solution was filtered through a short silica gel plug, reduced in volume to 3-5 mL, and treated with hexanes (50 mL). After 12 h at +5° C., the dark crystals were collected, washed with hexanes (3×5 mL), and dried under vacuum. The yield was 4.58 g (72%), m.p. 186-187° C. The structure was confirmed by single-crystal X-ray diffraction.

### Example 20

Bis(2-pyrrolylald(m-trifluoromethylphenyl)imino)copper(II). A mixture of 2-formylpyrrole (1.00 g), water (25 mL), 3-trifluoromethylaniline (1.85 g), and copper (II) acetate (1.00 g) was vigorously stirred for 3 days. The solid was filtered, washed with water, air-dried on the filter, and dissolved in dichloromethane. The solution was filtered through a short silica gel plug and evaporated to dryness. The oily residue was re-dissolved in MeOH (10 mL). After 1.5 h at +5° C., the dark-brown crystals were collected, washed with cold MeOH, and dried. Hexanes (10 mL) was added. After 1 h at +5° C., the crystals were separated and dried under vacuum. The yield was 1.55 g (55%). Anal. Calcd. for C₂₄H₁₆CuF₆N₄, %: C, 53.6; H, 3.0; N, 10.4. Found, %: C, 53.7; H, 3.0; N, 10.4. The structure was confirmed by single-crystal X-ray diffraction.

### Example 21

Bis(2-pyrrolylaldbenzylimino)copper(II). A mixture of 2-formylpyrrole (2.08 g), water (20 mL), and benzylamine (2.60 g) was stirred for 10 min. Copper (II) acetate (2.20 g) was added, and the mixture was stirred for 0.5 h. Water (20 mL) was added, and the stirring continued for 1.5 h. The resulting solid was filtered, washed with water, air-dried on the filter, and dissolved in dichloromethane. The solution was filtered through a short silica gel plug and evaporated to leave a dark viscous oil. Hexanes (50 mL) were added to the residue and the mixture was kept at +5° C. for 2 h. The dark crystals were collected, washed with hexanes, and dried under vacuum. The yield was 2.80 g (60%). Anal. Calcd. for C₂₄H₂₂CuN₄, %: C, 67.0; H, 5.2; N, 13.0. Found, %: C, 67.0; H, 5.2; N, 13.0.

### Example 22

Bis(2-pyrrolylald(2-ethoxycarbonylethyl)imino)copper(II). A mixture of 2-formylpyrrole (0.62 g), water (15 mL), β-alanine ethyl ester hydrochloride (1.50 g), and copper (II) acetate (0.65 g) was stirred for 5 min. A solution of NaOH (0.6 g) in water (5 mL) was added and the mixture was stirred for 3 hours to produce brown oil. Hexanes (30 mL) was added and agitation resumed for 30 min. After evaporation of hexanes the mixture was extracted with dichloromethane (4×10 mL). The combined dichloromethane extracts were filtered through a short silica gel plug and evaporated. The dark oily residue was stirred with hexanes (20 mL), after which the volatiles were evaporated under vacuum to produce brown oil. The latter crystallized upon standing. Yield: 0.80 g (54%). Anal. Calcd. for C₂₀H₂₆CuN₄O₄, %: C, 53.4; H, 5.8; N, 12.4. Found, %: C, 53.1; H, 5.4; N, 12.3.

### Example 23

Bis(2-pyrrollald(2-isopropoxycarbonylethyl)imino)copper)(II). A mixture of 2-formylpyrrole (1.65 g), water (30 mL), β-alanine isopropyl ester hydrochloride (3.80 g; prepared as described in Inorg. Chem., 1986, vol. 25, p. 1956), and copper (II) acetate (1.90 g) was stirred for 10 min. A solution of NaOH (1.60 g) in water (10 mL) and hexanes (40 mL) were added, and the mixture was stirred for 1.5 hours. The upper hexane layer was separated by a pipette and disposed of. The mixture was extracted with dichloromethane (2×40 mL, then 4×10 mL). The combined extracts were filtered through a short silica gel plug and evaporated. The residue was dissolved in ca. 3 mL of dichloromethane and hexanes (50 mL) added. After 5 hours at +5° C. the product precipitated in the form of well-shaped brown crystals which were separated, washed with hexanes, and dried under vacuum. The yield was 2.85 g (69%). Anal. Calcd. for C₂₂H30CuN₄O₄, %: C, 55.3; H, 6.3; N, 11.7. Found, %: C, 55.3; H, 6.1; N, 11.9.

### Example 24

Bis(2-acetylpyrrolyl)copper(II). A mixture of acetylpyrrole (2.04 g), copper (II) acetate (2.04 g), and water (75 mL) was stirred at room temperature in air for 1 h. A solution of NaOH (0.83 g) in water (10 mL) was added and the stirring was continued for 2 more hours. The resulting solid was filtered, washed with water, dried, and dissolved in dichloromethane. After filtration, the solid-free dark green solution was reduced in volume to ca. 10 mL and treated with hexanes (100 mL). The mixture was kept at about +5° C. overnight. The resulting green crystals were separated by decantation, washed with hexanes, and dried under vacuum. The yield was 1.74 g (67%), m.p. 205° C. The structure was confirmed by single-crystal X-ray diffraction.

### Example 25

Bis(2-pyrrolylaldmethylimino)copper(III). Two batches of the crude complex were prepared separately. In the first batch, to a vigorously stirring mixture of 2-formylpyrrole (20.0 g), CuCl₂.2H₂O (19.7 g; 10% excess), and water (200 mL), was added 40% aqueous methylamine (20 mL) and after ca. 1 minute a solution of NaOH (10.6 g) in water (100 mL). A black-brown precipitate formed quickly. After the mixture was stirred for 2 hours, the product was separated by filtration, washed with water, and dried in air. The 2nd batch was prepared similarly, using 2-formylpyrrole (25.0 g), CuCl₂.2H₂O (24.6 g) in water (200 mL), 40% aqueous methylamine (25 mL), and a solution of NaOH (16.6 g) in water (150 mL). The two crude products were combined and purified by Soxhlet extraction with simultaneous filtration through silica gel in a double thimble setup. The product was placed in the inner thimble which was centered inside a larger thimble. The space in between the two thimbles was filled with silica gel. After the extraction was complete (extracts colorless) the mixture in the receiver was reduced in volume to ca. 150 mL, treated with hexanes (500 mL), and kept in an ice bath for 2 h. The black-brown crystals were separated, washed with hexanes (3×50 mL), and dried under vacuum to give 50.5 g of the pure product. Evaporation of the combined mother liquor and washings, followed by recrystallization of the residue from dichloromethane-hexanes afforded additional 6.4 g of the pure complex. Total yield: 56.9 g (86%). Anal. Calcd. for C₁₂H₁₄CuN₄, %: C, 51.9; H, 5.1; N, 20.2. Found, %: C, 51.9; H, 4.9; N, 20.2.

### Example 26

Bis(2-pyrrolylaldethylimino)copper(II). To a vigorously stirring mixture of 2-formylpyrrole (45.4 g), CuCl₂.2H₂O (44.7 g), and water (450 mL), was added 70% aqueous methylamine (50 mL) and then a solution of NaOH (24.0 g) in water (100 mL). After the mixture was stirred for 3 hours, the black-brown product was separated by filtration, washed with water, and dried. The crude product was dissolved in dichloromethane (500 mL), and the solution was filtered through a short silica gel plug which was then washed with dichloromethane. The combined filtrate and washings were evaporated to a thick paste of the complex in a small amount of dichloromethane, and treated with hexanes (300 mL). After 2 h at +5° C. the black crystals were separated, washed with hexanes (3×50 mL), and dried under vacuum. The yield of the analytically pure complex was 68.0 g (94%). Anal. Calcd. for C₁₄H₁₈CuN₄, %: C, 55.0; H, 5.9; N, 18.3. Found, %: C, 55.1; H, 5.9; N, 18.2.

### Volatility of the Copper Complexes

All solid Cu(II) complexes described above sublime under vacuum without decomposition. Bis(2-pyrrolylald(2-ethylhexyl)imino)copper(II), which is an oil at ambient temperature, can be distilled under reduced pressure. Conditions for vacuum deposition of the compounds vary depending on the nature of the substituent on the ligand. For example, in the pressure range of 0.02-1 torr, bis(2-pyrrolylaldalkylimino)copper(II) complexes (alkyl=Me, Et, i-Pr, Pr, Bu) sublime cleanly at 85-120° C. Bis(2-pyrrolylaldphenylimino)copper(II) sublimes at ca. 150-160° C./0.02-0.1 torr.

### Deposition of Copper Metal Films

### Example 27

Diethylsilane as the Reducing Agent. Bis(2-pyrrolylaldphenylimino)copper(II) (10 mg) and diethylsilane (0.2 mL) were placed in a glass tube under nitrogen. The tube was sealed and then gradually heated to 210° C. At 160-170° C. and above, copper metal formation (thin film) was noticed on the inner walls of the tube.

### Example 28

Ammonia-Hydrogen as the Reducing Agent. A bis(2-pyrrolylaldalkylimino)copper(II) complex (3-10 mg) was placed in a glass tube. The tube was heated under a mixture of NH₃ and H₂ (ca. 1:1) with the temperature gradually being raised from 120° C. to 220° C. At 180° C. and above, the formation of copper metal occurred, in the form of a thin film on the inner wall of the tube. Bis(2-pyrrolylaldmethylimino)copper(II) and bis(2-pyrrolylaldethylimino)copper(II) gave the best films. For copper metal deposition, ammonia-hydrogen mixtures were superior to either pure component. Bis(2-acetylpyrrolyl)copper(II) underwent reduction to copper metal with ammonia-hydrogen at a slightly lower temperature, around 160° C.

What is claimed is:

1. A process for preparing pyrrolealdimines, comprising the steps of:
a) reacting 2-formylpyrrole with a primary amine, RNH₂, in an aqueous solution, wherein the temperature is about 5 to about 30° C., and R is C₁ to C₁₀ alkyl or substituted alkyl, or C₆ to C₁₂ aryl or substituted aryl;
b) adding a water-immiscible organic compound to form an aqueous phase and an organic phase; and
c) isolating the organic phase.

2. The process of claim 1 wherein the water-immiscible organic compound is selected from the group consisting of alkanes, chlorinated alkanes, cycloalkanes, and aromatic solvents.

3. The process of claim 2 wherein the water-immiscible compound is selected from the group consisting of pentane, hexanes, heptanes, chloroform, dichloromethane, carbon tetrachloride, cylcopentane, cyclohexane, benzene, and toluene.

4. The process of claim 1, wherein the temperature is from about 10° C. to about 25° C.

5. The process of claim 1, wherein in step (a) an aqueous solution of the primary amine, RNH₂, is added to 2-formylpyrrole, and R is methyl or ethyl.

6. The process of claim 1, wherein in step (a) the primary amine is added to a mixture of 2-formylpyrrole in water, and R is propyl, isopropyl, butyl, isobutyl, or sec-butyl.

7. The process of claim 1, wherein in step (a), tert-butylamine is added to 2-formylpyrrole.

8. An aqueous process for preparing Cu(II) complexes of 2-pyrrole ligands comprising reacting an aqueous mixture of 2-formylpyrrole, a primary amine, R¹NH₂, and a source of Cu(II), wherein
R¹ is selected from the group consisting of C₁-C₁₀ alkyl or substituted alkyl; C₆ to C₁₂ aryl or substituted aryl; allyl; benzyl; NHR³; and NR⁴R⁵; and
R³, R⁴, and R⁵ are independently selected from the group of C₁-C₆ alkyl or substituted alkyl, and C₆ to C₁₂ aryl or substituted aryl.

9. The process of claim 8, wherein the primary amine is selected from the group consisting of methylamine, ethylamine, propylamine, isopropylamine, n-butylamine, t-butylamine, isobutylamine, 2-ethylhexylamine, aniline, 3-trifluoromethylaniline, β-alanine isopropyl ester, β-alanine ethyl ester and benzylamine.

10. An aqueous process for preparing Cu(II) complexes of 2-pyrrole ligands comprising
a. reacting 2-formylpyrrole and a primary amine in water; and
b. adding a source of copper(II) and allowing the mixture to react to form the copper(II) complex.

11. The process of claim 10, wherein the source of copper(II) is selected from the group consisting of copper hydroxide, copper(II) chloride, copper nitrate, copper sulfate, copper(II) salts of carboxylic acids, and copper alkoxides.

12. An aqueous process for preparing Cu(II) complexes of a 2-acylpyrrole comprising the steps of
a. contacting an aqueous mixture of a source of Cu(II) with a 2-acylpyrrole, 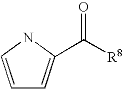
where R⁸ is C₁ to C₁₀ alkyl; and
b. further reacting the aqueous mixture with a base.

13. The process of claim 12, wherein the 2-acylpyrrole is 2-acetylpyrrole.

14. A Cu(II) complex comprising:
a) a copper atom; and
b) two pyrrole ligands bound to said copper atom, wherein said pyrrole ligands are independently selected from the group consisting of 2-pyrroleald-n-propylimino, 2-pyrroleald-1-butyl-imino, 2-pyrroleald-n-butyl-imino, 2-pyrroleald-2-ethylhexyl-imino, 2-pyrroleald-m-trifluoromethylphenyl-imino, 2-pyrrolylald(2-isopropoxycarbonylethyl)imino, 2-pyrrolylald(2-ethoxycarbonylethyl)imino and 2-pyrroleald-benzyl-imino ligands.

15. A Cu(II) complex selected from the group consisting of bis(2-pyrrolylald-n-propylimino)copper(II), bis(2-pyrrolylald-n-butylimino)copper(II), bis(2-pyrrolylaldisobutylimino)copper(II), bis(2-pyrrolylald(2-ethylhexyl)imino)copper(II), bis(2-pyrrolylald(m-trifluoromethylphenyl)imino)copper(II), bis(2-pyrrolylaldbenzylimino)copper(II), bis(2-pyrrolylald(2-ethoxycarbonylethyl)imino)copper(II), bis(2-pyrrolylald(2-isopropoxycarbonylethyl)imino)copper(II), and bis(2-acetylpyrrolyl)copper(II).

16. A process for depositing copper on a substrate comprising:
a) adsorbing onto a substrate at least one Cu(II) complex of structure 1, 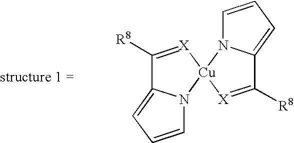
wherein:
X is O, and R⁸ is C₁-C₁₀ alkyl or substituted alkyl, or C₆ to C₁₂ aryl or substituted aryl; or
X is NR¹ and R⁸ is H;
R¹ is selected from the group consisting of C₁-C₁₀ alkyl or substituted alkyl; C₆ to C₁₂ aryl or substituted aryl; allyl; benzyl; NHR³; and NR⁴R⁵; and
R³, R⁴, and R⁵ are independently selected from the group of C₁-C₆ alkyl or substituted alkyl, and C₆ to C₁₂ aryl or substituted aryl; and
b) exposing said absorbed complex to a reducing agent to form copper metal.

17. The process of claim 16, wherein the Cu(II) complex is selected from the group consisting of bis(2-pyrrolealdmethylimino)copper(II), bis(2-pyrrolealdethylimino)copper(II), bis(2-pyrroleald-iso-propylimino)copper(II), bis(2-pyrroleald-t-butylimino)copper(II), bis(2-pyrrolealdphenylimino)copper(II), bis(2-pyrrolylald-n-propylimino)copper(II), bis(2-pyrrolylald-n-butylimino)copper(II), bis(2-pyrrolylaldisobutylimino)copper(II), bis(2-pyrrolylald(m-trifluoromethylphenyl)imino)copper(II), bis(2-pyrrolylaldbenzylimino)copper(II), and bis(2-acetylpyrrolyl)copper(II), and wherein the substrate is selected from the group consisting of glass, metals and ceramics, and silicon wafers coated with a barrier layer, and wherein the reducing agent is selected from the group consisting of ammonia, ammonia/hydrogen mixtures, hydrazine, CO/hydrogen mixtures, 9-BBN, borane, dihydrobenzofuran, pyrazoline, diethylsilane, dimethylsilane, ethylsilane, phenylsilane, and silane.

18. A process for depositing copper on a substrate comprising heating a reducing agent and at least one Cu(II) complex of structure 1, 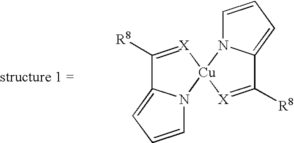
in the presence of a substrate, wherein:
X is O, and R⁸ is C₁-C₁₀ alkyl or substituted alkyl, or C₆ to C₁₂ aryl or substituted aryl; or
X is NR¹ and R⁸ is H;
R¹ is selected from the group consisting of C₁-C₁₀ alkyl or substituted alkyl; C₆ to C₁₂ aryl or substituted aryl; allyl; benzyl; NHR³; and NR⁴R⁵; and
R³, R⁴, and R⁵ are independently selected from C₁-C₆ alkyl or substituted alkyl, and C₆ to C₁₂ aryl or substituted aryl.

19. An article comprising a substrate with a Cu(II) complex of structure 1 adsorbed on the surface or in or on porosity in the substrate, 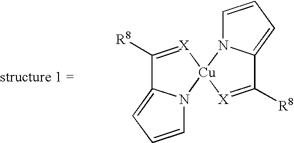
wherein:
X is O, and R⁸ is C₁-C₁₀ alkyl or substituted alkyl, or C₆ to C₁₂ aryl or substituted aryl; or
X is NR¹ and R⁸ is H;
R¹ is selected from the group consisting of C₁-C₁₀ alkyl or substituted alkyl; C₆ to C₁₂ aryl or substituted aryl; allyl; benzyl; NHR³; and NR⁴R⁵; and
R³, R⁴, and R⁵ are independently selected from C₁-C₆ alkyl or substituted alkyl, and C₆ to C₁₂ aryl or substituted aryl.

20. An article of claim 19, wherein the substrate is selected from the group consisting of glass, metals and ceramics, and silicon wafers coated with a barrier layer.
